(19) United States
(12) Patent Application Publication
(10) Pub. No.: US 2006/0264381 A1
(43) Pub. Date: Nov. 23, 2006

(54) Methods of treating obsessive compulsive disorder

(76) Inventor: Mark F. Bear, Bristol, RI (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/418,815

(22) Filed: May 5, 2006

Related U.S. Application Data
(60) Provisional application No. 60/677,918, filed on May 5, 2005.

(51) Int. Cl. A61K 38/04; A61K 31/401; A61K 31/198

(52) U.S. Cl. 514/19; 514/423; 514/567

(57) ABSTRACT

An subjects with obsessive compulsive disorder is treated. In one embodiment, the subject is administered a compound down regulates Group I mGluR signaling. In another embodiment, the subject is administered a compound that down regulates endocannabinoid signaling. The subject that has obsessive compulsive disorder can further have at least one condition selected from the group consisting of fragile X syndrome, autism and mental retardation.

### RELATED APPLICATIONS

This application claims the benefit of U.S. Provisional Application No. 60/677,918, filed May 5, 2005. The teachings of the above application are incorporated herein by reference.

### GOVERNMENT SUPPORT

The invention was supported, in whole or in part, by a grant 5-R01-HD46943 from The National Institute of Child Health and Human Development. The Government has certain rights in the invention.

### BACKGROUND OF THE INVENTION

Obsessive compulsive disorder (OCD) affects approximately 3.3 million Americans (National Institutes of Mental Health). Currently available treatments for OCD include cognitive-behavioral psychotherapy (CBT) alone or in combination serotonin reuptake inhibitor (SRI) or selective serotonin reuptake inhibitor (SSRI) treatment. However, acceptance and compliance with CBT is often difficult and specific CBT techniques may need to be matched with specific compulsions or obsessions. In addition, treatment with SRIs and SSRIs alone may be ineffective in eliminating OCD symptoms, must be closely monitored in subjects, particularly adolescents, and can be frequently associated with adverse side effects, such as nervousness, insomnia, sedation, dizziness, weight gain, irregular heart beats, alterations in blood pressure and nausea. Thus, there is a need to develop new, improved and effective methods of treatment for obsessive compulsive disorder.

### SUMMARY OF THE INVENTION

The present invention is directed to a method of treating obsessive compulsive disorder in a subject.

In one embodiment, the method comprising the step of administering to a subject having obsessive compulsive disorder at least one compound that down regulates Group I mGluR signaling.

In another embodiment, the method comprising the step of administering to a subject having obsessive compulsive disorder at least one compound that down regulates endocannabinoid signaling.

Advantages of the claimed invention include, for example, the treatment of OCD in a subject in a manner that can be acceptable to the subject, is easy for the subject to comply with, is effective in treating a range of obsessions and compulsions, and is without significant side effects associated with SRIs and SSRIs. The claimed method provides an efficient way to treat and reduce the severity of obsessions and compulsions associated with OCD.

Thus, treatment with compounds that down regulate Group I mGluR signaling or endocannabinoid signaling in a subject having OCD can potentially halt, diminish or reverse the symptoms (obsessions and compulsions) associated with OCD, thereby increasing the quality of life of the subject.

### BRIEF DESCRIPTION OF THE FIGURE

FIG. 1 depicts the distribution of Group I (Gp1) mGluR protein in the rat brain.

FIG. 2 depicts the down regulation of Group I mGluR (mGluR1 and mGluR5) signaling to thereby treat obsessive compulsive disorder.

FIG. 3 depicts down regulation of endocannabinoid signaling to thereby treat obsessive compulsive disorder.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

The present invention is directed to a method of treating obsessive compulsive disorder in a subject. The subject with obsessive compulsive disorder being treated by the methods of the invention can also have at least one condition selected from the group consisting of fragile X syndrome, autism and mental retardation.

As previously reported, Group I mGluR agonists increase long term depression (LTD) in brains obtained from fragile X mental retardation protein (FMRP) knockout (KO) mice (Huber, K. M., et al., Proc. Natl. Acad. Sci. USA). Elevated or irregular LTD (LTD not within a normal range), in particular striatum LTD, may be associated with OCD. Thus, it is believed that down regulation of Group I mGluR can attenuate OCD which, like elevated LTD, has been observed with fragile X syndrome.

In one embodiment, the invention is a method of treating a subject comprising the step of administering at least one compound that down regulates Group I mGluR signaling in a subject having obsessive compulsive disorder. mGluR1 signaling or mGluR5 signaling, alone or in combination, can be down regulated.

The subject treatment by the methods of the invention described herein can be a rodent (e.g., mouse, rat) or a primate (e.g., a monkey, baboon, human). In a particular embodiment, the subject is a human.

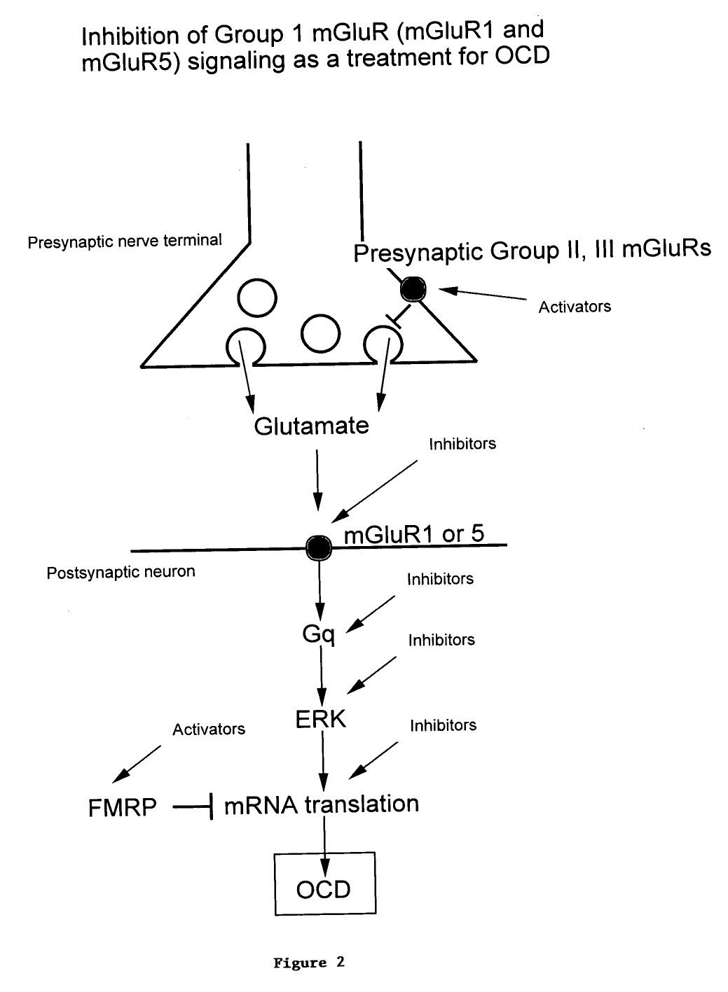

“Down regulates mGluR signaling,” as used herein, refers to any decrease or any inhibition in a cellular process or a cellular event or intermediate in a cellular event associated with any mechanism whereby metabotropic glutamate receptors (mGluR5, namely Group I, II or III mGluR5) mediate a biological response. For example, down regulation can be the prevention or any decrease in binding of a signal external to a cell (a first messenger), such as a ligand (e.g., glutamate), to a Group I mGluR. Down regulation can be disruption of a cellular process following binding of an external signal (e.g., ligand) to an mGluR, such as the prevention of activation of adenylyl cyclase or phospholipase C (PLC). Down regulation can also be disruption of a cellular processes following binding of an external signal to Group I mGluR, such as the prevention of activation of a G-protein (Gs, Gq), a decrease in a G-protein (Gs, Gq) activation, prevention of activation of second messengers activated by Group I mGluR (e.g., cAMP, IP₃, diacylglycerol (DAG)) or a decrease in the activity of an intracellular effector, such as a cAMP-dependent protein kinase, protein kinase C (PKC) or calcium release. FIG. 2 depicts down regulation of mGluR signaling and, thereby, treatment of OCD.

Down regulation of Group I mGluR signaling can also be a decrease or inhibition in the release of glutamate as shown, for example, in Figures and 3. A decrease or inhibition of glutamate release can be through activation of presynaptic Group II and/or Group III mGluR5 by, for example, agonists of Group II and Group III mGluR5 (FIG. 2). An “agonist,” as used herein, is a compound that activates cell signaling. Exemplary Group II and Group III mGluR agonists for use in the invention include LY354740, L-AP4 (Capogna, M. Eur. J. Neurosci. 19: 2847-2858 (2004), the teachings of which are hereby incorporated by reference in its entirety) (2R,4R)-4-Aminopyrrolidine-2,4-dicarboxylate ((2R,4R)-APDC), (2S,1′S,2′ S)-2-(Carboxycyclopropyl)glycine (L-CCG-1), N-Acetyl-L-aspartyl-L-glutamic acid (Spaglumic acid), (S)-3-Carboxy-4-hydroxyphenylglycine ((S)-3C4HPG), (S)-4-Carboxy-3-hydroxyphenylglycine ((S)-4C3HPG) and AMN 082 dihydrochloride (Flor et al., Neuropharmacology 49:244 (2005), the teachings of which are hereby incorporated by reference in its entirety).

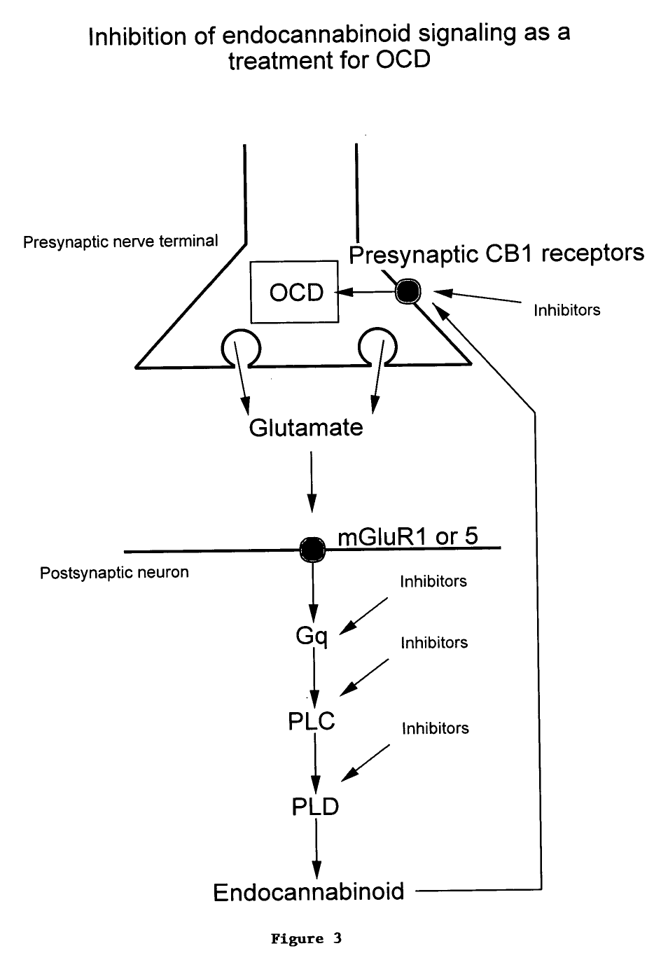

Excessive LTD can be decreased or inhibited by, for example, inhibiting presynaptic CB1 receptors. AM251 is one embodiment of a factor suitable for this purpose (Rouach, N., et al., Eur. J. Neurosci. 18:1017-1020 (2003), the teachings of which are hereby incorporated by reference in its entirety). FIG. 3 depicts inhibition of presynaptic CB1 receptors, thereby treating OCD.

Group I mGluR signaling can also be down regulated by decreasing or inhibiting intracellular signals or intermediates generated in response to postsynaptic activation by Group I mGluR (FIGS. 2 and 3), by, for example, by administering U0126 (Gallagher, et al., J. Neurosci. 24:4859-4864 (2004), the teachings of which are hereby incorporated by reference in its entirety) to a subject with OCD.

Group I mGluR signaling also can be down regulated by decreasing or inhibiting protein synthesis in response to mGluR activation with, for example, rapamycin (Bierer et al., PNAS 87:9231 (1990), the teachings of which are hereby incorporated by reference in its entirety). The protein synthesis in response to mGluR activation can be decreased or inhibited by decreasing or inhibiting the transcription, translation, posttranslational modifications, intracellular half-life and/or intracellular processing of signals mediated or activated by mGluR (FIG. 2).

In a specific embodiment, suitable compounds that down regulate Group I mGluR can be Group I mGluR antagonists. An mGluR antagonist is a substance which diminishes or abolishes the effect of a ligand (or agonist) that activates an mGluR. Thus, the antagonist can be, for example, a chemical antagonist, a pharmacokinetic antagonist, an antagonist by receptor block, a non-competitive antagonist, or a physiological antagonist.

Antagonists may act at the level of the ligand-receptor interactions, such as by competitively or non-competitively (e.g., allosterically) inhibiting ligand binding. The antagonist can act downstream of the receptor, such as by inhibiting receptor interaction with a G protein or downstream events associated with G protein activation, such as stimulation of phospholipase C, elevation in intracellular calcium, the production of or levels of cAMP or adenylcyclase, stimulation and/or modulation of ion channels (e.g., K+, Ca++). The antagonists can alter, diminish, halt, inhibit or prevent the above-referenced cellular signaling events.

A “pharmacokinetic antagonist” reduces the concentration of drug or ligand at its site of action, e.g., by increasing the rate of metabolic degradation of the ligand or drug. Antagonism by receptor-block can be reversible competitive antagonism, and irreversible, or non-equilibrium, competitive antagonism. Reversible competitive antagonism occurs when the rate of dissociation of the antagonist molecule from the receptor is sufficiently high that, on addition of the ligand, the antagonist molecules binding the receptors are effectively replaced by the ligand. Irreversible or non-equilibrium competitive antagonism occurs when the antagonist dissociates very slowly or not at all from the receptor, with the result that no change in the antagonist occupancy takes place when the ligand is applied. A “competitive antagonist,” as used herein, is a molecule which binds directly to the receptor or ligand in a manner that sterically interferes with the interaction of the ligand with the receptor.

Non-competitive antagonism occurs when an antagonist does not compete directly with ligand binding at the receptor, but instead blocks a point in the signal transduction pathway subsequent to receptor activation by the ligand. An antagonist can also be a substance that diminishes or abolishes expression of functional mGluR. For example, an antagonist can be a compound that diminishes or abolishes the expression of the gene encoding mGluR1 or mGluR5, the translation of mGluR1 or mGluR5 RNA, the post-translational modification of mGluR1 or mGluR5 protein, or the insertion of mGluR1 mGluR5 into the cell membrane.

Examples of subjects that can benefit from the methods of the invention includes mammals, such as human and non-human primates and companion animals such as dogs and cats. In a particular embodiment, the subject is a human.

Examples of suitable antagonists of mGluR5 are described in WO 01/66113, WO 01/32632, WO 01/14390, WO 01/08705, WO 01/05963, WO 01/02367, WO 01/02342, WO 01/02340, WO 00/20001, WO 00/73283, WO 00/69816, WO 00/63166, WO 00/26199, WO 00/26198, EP-A-0807621, WO 99/54280, WO 99/44639, WO 99/26927, WO 99/08678, WO 99/02497, WO 98/45270, WO 98/34907, WO 97/48399, WO 97/48400, WO 97/48409, WO 98/53812, WO 96/15100, WO 95/25110, WO 98/06724, WO 96/15099 WO 97/05109, WO 97/05137, U.S. Pat. No. 6,218,385, U.S. Pat. No. 5,672,592, U.S. Pat. No. 5,795,877, U.S. Pat. No. 5,863,536, U.S. Pat. No. 5,880,112, U.S. Pat. No. 5,902,817, all of which are incorporated by reference in their entirety.

Different classes of mGluR5 antagonists are described in WO 01/08705 (pp. 3-7), WO 99/44639 (pp. 3-11), and WO 98/34907 (pp. 3-20).

Suitable mGluR5 antagonists are also described in WO 01/02367 and WO 98/45270 and have the formula:

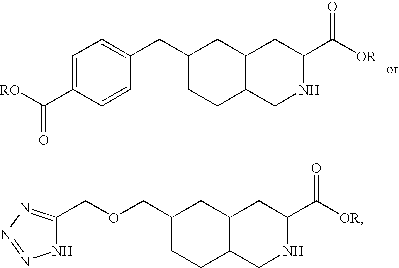

wherein R represents H or a hydrolyzable hydrocarbon moiety such as an alkyl, heteroalkyl, alkenyl, or aralkyl moiety.

In certain such embodiments, the isoquinoline system has the stereochemical array 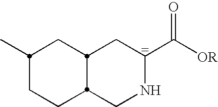
(wherein, as is known in the art, a dark spot on a carbon indicates hydrogen coming out of the page, and a pair of dashes indicates a hydrogen extending below the plane of the page), the enantiomer thereof, of a racemic mixture of the two.

Another class of antagonists, described in WO 01/66113, has the formula:

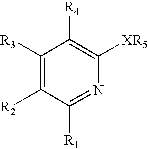

wherein

R₁ denotes hydrogen, lower alkyl, hydroxyl-lower alkyl, lower alkyl-amino, piperidino, carboxy, esterified carboxy, amidated carboxy, unsubstituted or lower alkyl-, lower alkoxy-, halo- and/or trifluoromethyl-substituted N-lower-alkyl-N-phenylcarbamoyl, lower alkoxy, halo-lower alkyl or halo-lower alkoxy;

R₂ denotes hydrogen, lower alkyl, carboxy, esterified carboxy, amidated carboxy, hydroxyl-lower alkyl, hydroxyl, lower alkoxy or lower alkanoyloxy, 4-(4-fluoro-benzoyl-piperidin-1-ylcarboxy, 4-t.butyloxycarbonyl-piperazin-1-yl-carboxy, 4-(4-azido-2-hydroxybenzoyl)-piperazin-1-yl-carboxy or 4-(4-azido-2-hydroxy-3-iodo-benzoyl)-piperazin-1-yl-carboxy;

R₃ represents hydrogen, lower alkyl, carboxy, lower alkoxy-carbonyl, lower alkyl-carbamoyl, hydroxy-lower alkyl, di-lower alkyl-aminomethyl, morpholinocarbonyl or 4-(4-fluoro-benzoyl)-piperadin-1-yl-carboxy;

R₄ represents hydrogen, lower alkyl, hydroxy, hydroxy-lower alkyl, amino-lower alkyl, lower alkylamino-lower alkyl, di-lower alkylamino-lower alkyl, unsubstituted or hydroxy-substituted lower alkyleneamino-lower alkyl, lower alkoxy, lower alkanoyloxy, amino-lower alkoxy, lower alkylamino-lower alkoxy, di-lower alkylaino-lower alkoxy, phthalimido-lower alkoxy, unsubstituted or hydroxy-or-2-oxo-imidazolidin-1-yl-substituted lower alkyleneamino-lower alkoxy, carboxy, esterified or amidated carboxy, carboxy-lower alkoxy or esterified carboxy-lower alkoxy; and

X represents an optionally halo-substituted lower alkenylene or alkynylene group bonded via vicinal saturated carbon atoms or an azo (—N═N—) group, and R₅ denotes an aromatic or heteroaromatic group which is unsubstituted or substituted by one or more substituents selected from lower alkyl, halo, halo-lower alkyl, halo-lower alkoxy, lower alkenyl, lower alkynyl, unsubstituted or lower alkyl-, lower alkoxy-, halo-and/or trifluoromethyl-substituted phenyl, unsubstituted or lower alkyl-, lower alkoxy-, halo and/or trifluoromethyl-substituted phenyl-lower alkynyl, hydroxy, hydroxy-lower alkyl, lower alkanoyloxy-lower alkyl, lower alkoxy, lower alkenyloxy, lower alkylenedioxy, lower alkanoyloxy, amino-, lower alkylamino-, lower alkanoylamino- or N-lower alkyl-N-lower alkanoylamino-lower alkoxy, unsubstituted or lower alkyl-, lower alkoxy-, halo- and/or trifluoromethyl-substituted phenoxy, unsubstituted or lower alkyl-, lower alkoxy-, halo and/or trifluoromethyl-substituted phenyl-lower alkoxy, acyl, carboxy, esterified carboxy, amidated carboxy, cyano, carboxy-lower alkylamino, esterified carboxy-lower alkylamino, amidated carboxy-lower alkylamino, phosphono-lower alkylamino-esterified phosphono-lower alkylamino, nitro, amino, lower alkylamino, di-lower alkylamino-acylamino, N-acyl-N-lower alkylamino, phenylamino, phenyl-lower alkylamino, cycloalkyl-lower alkylamino or heteroaryl-lower alkylamino each of which may be unsubstituted or lower alkyl-, lower alkoxy-, halo- and/or trifluoromethyl-substituted; their N-oxides and their pharmaceutically acceptable salts.

As described in WO 01/66113 and WO 00/20001, the Group I mGluR antagonist can have the formula: 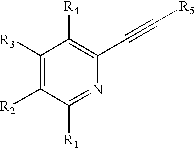
wherein

R₁ is hydrogen, (C₁₋₄)alkyl, (C₁₋₄)alkoxy, cyano, ethynyl or di(C₁₋₄)alkylamino,

R₂ is hydrogen, hydroxy, carboxy, (C₁₋₄) alkoxycarbonyl, di(C₁₋₄)alkylaminomethyl, 4-(4-fluoro-benzoyl)-piperidin-1-yl-carboxy, 4-t-butyloxycarbonyl-piperazin-1-yl-carboxy, 4-(4-azido-2-hydroxybenzoyl)-piperazin-1-yl-carboxy, or 4-(4-azido-2-hydroxy-3-iodo-benzoyl)-piperazin-1-yl-carboxy,

R₃ is hydrogen, (C₁₋₄)alkyl, carboxy, (C₁₋₄)alkoxycarbonyl, (C₁₋₄)alkylcarbamoyl, hydroxy(C₁₋₄)alkyl, di(C₁₋₄)alkylaminomethyl, morpholinocarbonyl or 4-(4-fluoro-benzoyl)-piperazin-1-yl-carboxy,

R₄ is hydrogen, hydroxyl, carboxy, C(₂₋₅)alkanoyloxy, (C₁₋₄)alkoxycarbonyl, amino (C₁₋₄)alkoxy, di(C₁₋₄)alkylamino(C₁₋₄)alkoxy, di(C₁₋₄)alkylamino(C₁₋₄)alkyl or hydroxy(C₁₋₄)alkyl, and

R₅ is a group of formula: 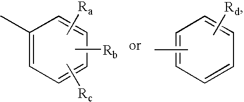
wherein

Rₐ and R_{b} independently are hydrogen, halogen, nitro, cyano, (C₁₋₄)alkyl, (C₁₋₄)alkoxy, trifluoromethyl, trifluoromethoxy or (C₂₋₅)alkynyl, and

R_{c} is hydrogen, fluorine, chlorine bromine, hydroxy-(C₁₋₄)alkyl, (C₂₋₅)alkanoyloxy, (C₁₋₄)alkoxy, or cyano, and

R_{d} is hydrogen, halogen or (C₁₋₄)alkyl;

in free form or in the form of pharmaceutically acceptable salts.

As described in WO 01/66113, mGluR5 antagonists can also have structures of the formula:

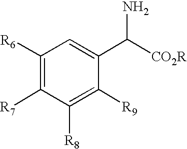

wherein R₆ is hydrogen, hydroxy, or C₁₋₆ alkoxy;

R₇ is hydrogen, carboxy, tetrazolyl, —SO₂H, —SO₃H, —OSO₃H, —CONHOH, or —P(OH)OR′, —PO(OH)OR′, —OP(OH)OR′ or —OPO(OH)OR′ where R′ is hydrogen, C₁₋₆ alkyl, C₂₋₆ alkenyl, or aryl C₁₋₆ aryl;
R₈ is hydrogen, hydroxy or C₁₋₄ alkoxy; and

R₉ is fluoro, trifluoromethyl, nitro, C₁₋₆ alkyl, C₃₋₇ cycloalkyl, C₂₋₆ alkenyl, C₂₋₆ alkynyl, C₁₋₆ alkylthio, heteroaryl, optionally substituted aryl, optionally substituted aryl C₁₋₆ alkyl, optionally substituted aryl C₂₋₆ alkenyl, optionally substituted aryl C₂₋₆ alkynyl, optionally substituted aryloxy, optionally substituted C₁₋₆ alkoxy, optionally substituted arythio, optionally substituted aryl C₁₋₆ alkylthio, —CONR″R′″, —NR″R′″, —OCONR″R′″ or —SONR″R′″, where R″ and R′″ are each hydrogen, C₁₋₆alkyl or aryl C₁₋₆ alkyl, or R″ and R′″ together form a C₃₋₇ alkylene ring;

or a salt or ester thereof.

Another class of mGluR5 antagonists is described in WO 00/63166 and has the formula:

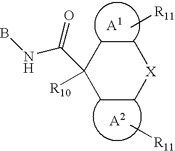

wherein

R₁₀ signifies hydrogen or lower alkyl;

R₁₁ signifies, independently for each occurrence, hydrogen, lower alkyl, lower alkoxy, halogen or trifluoromethyl;

X signifies O, S, or two hydrogen atoms not forming a bridge;

A¹/A² signify, independently from each other, phenyl or a 6-membered heterocycle containing 1 or 2 nitrogen atoms;

B is a group of formula 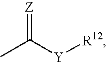
wherein

R¹² signifies lower alkyl, lower alkenyl, lower alkynyl, benzyl, lower alkyl-cycloalkyl, lower alkyl-cyano, lower alkyl-pyridinyl, lower alkyl-lower alkoxy-phenyl, lower alkyl-phenyl (optionally substituted by lower alkoxy), phenyl (optionally substituted by lower alkoxy), lower alkyl-thienyl, cycloalkyl, lower alkyl-trifluoromethyl, or lower alkyl-morpholinyl;

Y signifies —O—, —S— or bond;

Z signifies —O— or —S—;

or B is a 5-membered heterocyclic group of formulas

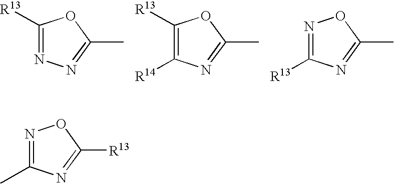

wherein

R¹³ and R¹⁴ independently signify hydrogen, lower alkyl, lower alkoxy, cyclohexyl, lower alkyl-cyclohexyl or trifluoromethyl, with the proviso that at least one of R¹³ or R¹⁴ is hydrogen;

as well as with their pharmaceutically acceptable salts.

Another class of mGluR1 antagonists is described in WO 01/32632 and has the formula: 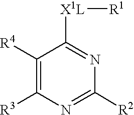
X¹ represents O or NH;
L represents a bond or a (1-6C) alkylene chain optionally interrupted by O, S, SO, SO or NH and optionally substituted on an alkylene carbon atom by fluoro, hydroxy, (1-4C)alkoxy or oxo;
R¹ represents an unsubstituted or substituted carbocyclic or heterocyclic group;
R² represents a hydrogen atom, a halogen atom, a carboxyl group, a cyano group, a SCH₂CN, or a group of formula X²—R⁵ in which X² represents a bond, O, S, SO, SO₂ or NH and R⁵ represents (1-8C)alkyl, (3-10C)cycloalkyl, halo(1-6C)alkyl, hydroxy(1-6C)alkyl, dihydroxy(1-4C)alkyl, (1-4C)alkoxy(1-4C)alkyl, (1-4C)alkanoyl(1-4C)alkyl, (1-4C)alkanoyloxy(1-4C)alkyl, carboxy(1-4C)alkyl, (1-4C)alkylaminocarbonyl(1-4C)alkyl, (1-4C)alkanoylamino, (1-4C)alkanoylamino(1-4C)alkyl, (1-4C)alkanoylamino[(1-4C)alkyl]₂, (1-4C)alkylthio(1-4C)alkyl, (1-4C)alkylsulfinyl(1-4C)alkyl, (1-4C)alkylsulfonyl(1-4C)alkyl, (1-4C)alkylsulfonylamino)(1-4C)alkyl, (1-4C)alkylamino-sulfonyl)(1-4C)alkyl, di(1-4C)alkylaminophosphonyl)(1-4C)alkyl, phenyl or phenyl(1-4C)alkyl in which any phenyl group is unsubstituted or substituted by one or two substituents selected independently from a halogen atom, (1-4C)alkyl and (1-4C)alkoxy; and
R³ and R⁴ each independently represents (1-4C)alkyl or together with the carbon atoms to which they are attached form an unsubstituted or substituted carbocyclic or heterocyclic ring;
   or a pharmaceutically acceptable salt thereof.

Another class of mGluR5 antagonists is described in WO 01/14390 and has the formula: 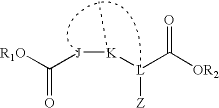
wherein,
either J and K are taken together with one or more additional atoms independently selected from the group consisting of C, O, S, and N in chemically reasonable substitution patterns to form a 3-7 membered saturated or unsaturated heterocyclic or carbocyclic ring, and L is —CH,
or J, K, and L are taken together with one or more additional atoms independently selected from the group consisting of C, O, S, and N in chemically reasonable substitution patterns to form a 4-8 membered saturated or unsaturated, mono-, bi-, or tricyclic, hetero- or carbocyclic ring structure;
Z is a metal chelating group;
R₁ and R₂ are independently hydrogen, C₁-C₉ alkyl, C₂-C₉ alkenyl, C₃-C₈ cycloalkyl, C₅-C₇ cycloalkenyl, or Ar, wherein each said alkyl, alkenyl, cycloalkyl, cycloalkenyl, or Ar is independently unsubstituted or substituted with one or more substituent(s); and
Ar is a carbocyclic or heterocyclic moiety which is unsubstituted or substituted with one or more substituent(s);
   or a pharmaceutically acceptable equivalent thereof.

mGluR5 antagonists are also described in U.S. Pat. No. 6,218,385 and have the formula: 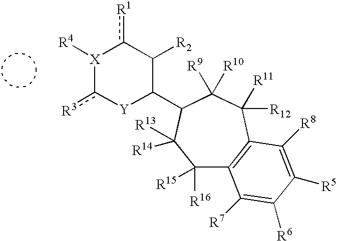
R¹ signifies hydrogen, hydroxy, lower alkyl, oxygen, halogen, or
—OR, —O(C₃-C₆)cycloalkyl, —O(CHR)ₙ—(C₃-C₆)cycloalkyl, —O(CHR)ₙ CN, —O(CHR)ₙ CF₃, —O(CHR)(CHR)ₙ NR₂, —O(CHR)(CHR)ₙOR, —O(CHR)ₙ-lower alkenyl, —OCF₃, —OCF₂—R, —OCF₂-lower alkenyl, —OCHRF, —OCHF-lower alkenyl, —OCF₂CRF₂, —OCF₂ Br, —O(CHR)ₙCF₂Br, —O(CHR)ₙ-phenyl, wherein the phenyl group may be optionally substituted independently from each other by one to three lower alkyl, lower alkoxy, halogen, nitro or cyano groups,
—O(CHR)(CHR)ₙ-morpholino, —O(CHR)(CHR)ₙ-pyrrolidino, —O(CHR)(CHR)ₙ-piperidino, —O(CHR) (CHR)ₙ-imidazolo, —O(CHR)(CHR)ₙ-triazolo, —O(CHR)ₙ-pyridino, —O(CHR)(CHR)ₙ—OSi-lower alkyl, —O(CHR)(CHR)ₙOS(O)₂-lower alkyl, —(CH₂)ₙCH═CF₂, —O(CHR)ₙ-2,2-dimethyl-[1,3]dioxolane, —O(CHR)ₙ—CHOR—CH₂OR, —O(CHR)ₙ—CHOR—(CHR)ₙ—CH₂OR or
—SR or —S(CHR)ₙCOOR, or
—NR₂, —N(R)(CHR)(CHR)ₙOR, —N(R)(CHR)ₙCF₃, —N(R)(CHR)(CHR)ₙ-morpholino, —N(R)(CHR)(CHR)ₙ-imidazolo, —N(R)(CHR)(CHR)ₙ-pyrrolidino, —N(R)(CiR)(CHR)ₙ-pyrrolidin-2-one, —N(R)(CHR)(CHR)ₙ-piperidino, —N(R)(CHR)(CHR)ₙ-triazolo, —N(R)(CHR)ₙ-pyridino, or
R¹ and R⁴ are interconnected to the groups—(CH₂)₃₋₅—, —(CH₂)₂—N═, —CH═N—N═—, —CH═CH—N═, —NH—CH═CH—or
—NR—CH₂—CH₂—and form together with any N or C atoms to which they are attached an additional ring;
n is 1-6;
R signifies hydrogen, lower alkyl or lower alkenyl, independently from each other, if more than one R is present;
R² signifies nitro or cyano;
R³ signifies hydrogen, lower alkyl, ═O, —S, —SR, —S(O)₂-lower alkyl, —(C₃-C₆)cycloalky or piperazino, optionally substituted by lower alkyl, or
   —CONR₂, —(CHR)ₙCONR₂, —(CHR)ₙOR, —(CH₂)ₙ—CF₃, —CF₃, —(CHR)ₙOC(O)CF₃, —(CHR)ₙ COOR, —(CHR)ₙ SC₆H₅, wherein the phenyl group may be optionally substituted independently from each other by one to three lower alkyl, lower alkoxy, halogen, nitro or cyano groups, —
   —(CHR)ₙ-1,3-dioxo-1,3-dihydro-isoindol, —(CHR)ₙ-tetrahydro-pyran-2-yloxy or —(CHR)ₙ—S-lower alkyl, or
   —NR₂, —NRCO-lower alkyl, —NRCHO, —N(R)(CHR)ₙCN, —N(R)(CHR)ₙCF₃, —N(R)(CHR)(CHR)ₙ—OR, —N(R)C(O)(CHR)ₙO-lower alkyl, —NR(CHR)ₙ-lower alkyl, —NR(CHR)(CHR)ₙ—OR, —N(R)(CHR)(CHR)ₙ—O-phenyl, wherein the phenyl group may be optionally substituted independently from each other by one to three lower alkyl, lower alkoxy, halogen, nitro or cyano groups,
   —N(R)(CHR)ₙ-lower alkenyl, —N(R)(CHR)(CHR)ₙ—O—(CHR)ₙOR, —N(R)(CHR)ₙC(O)O-lower alkyl, —N(R)(CHR)ₙC(O)NR-lower alkyl, —N(R)(CH₂)ₙ-2,2-dimethyl-[1,3]dioxolane, —N(R)(CHR)(CHR)ₙ morpholino, —N(R)(CHR)ₙ-pyridino, —N(R)(CHR)(CHR)ₙ-piperidino, —N(R)(CHR)(CHR)ₙ-pyrrolidino, —N(R)(CHR)(CHR)ₙ—O-pyridino, —N(R)(CHR)(CHR)ₙ imidazolo, —N(R)(CHR)ₙ—CR₂—(CHR)ₙ—OR, —N(R)(CHR)ₙ—CR₂—OR, —N(R)(CHR)ₙ—CHOR—CH₂OR, —N(R)(CHR)ₙ—CHOR—(CHR)ₙ—CH₂OR, or
   —OR, —O(CHR)ₙCF₃, —OCF₃, —O(CHR)(CHR)ₙ—O-phenyl, wherein the phenyl group maybe optionally substituted independently from each other by one to three lower alkyl, lower alkoxy, halogen, nitro or cyano groups,
   —O(CHR)(CHR)ₙ—O-lower alkyl, —O(CHR)ₙ-pyridino or
   —O(CHR)(CHR)ₙ-morpholino;
   or R³ and R⁴ are interconnected to the groups—(CH₂)₃₋₅—, —(CH₂)₂—N═, —CH═N—N═—, —CH═CH—N═, —NH—CH═CH—or
   NR—CH₂—CH₂—and form together with any N or C atoms to which they are attached an additional ring; and
R⁴ signifies hydrogen, lower alkyl, lower alkenyl or nitro, or
   —OR, —OCF₃, —OCF₂—R, —OCF₂-lower alkenyl, —OCHRF, —OCHF-lower alkenyl, —O(CHR)ₙCF₃, or
   —(CHR)ₙCHRF, —(CHR)ₙCF₂ R, —(CHR)ₙCF₃, —(C₃-C₆)cycloalkyl, —(CHR)ₙ(C₃-C₆)cycloalkyl, —(CHR)ₙCN, —(CHR)ₙ-phenyl, wherein the phenyl group may be optionally substituted independently from each other by one, to three lower alkyl, lower alkoxy, halogen, nitro or cyano groups,
   —(CHR)(CHR)ₙOR, —(CHR)ₙCHORCH₂OR; —(CHR)(CHR)ₙNR₂, —(CHR)ₙCOOR, —(CHR)(CHR)ₙOSi-lower alkyl,
   —(CHR)(CHR)ₙ—OS(O)₂-lower alkyl, —(CH₂)ₙ—CH═CF₂, —CF₃, —CF₂—R, —CF₂-lower alkenyl, —CHRF, —CHF-lower alkenyl, —(CHR)ₙ-2,2-dimethyl-[1,3]dioxolane, —(CH₂)ₙ-2-oxo-azepan-1-yl, —(CHR)(CHR)ₙ-morpholino, —(CHR)ₙ-pyridino, —(CHR)(CHR)ₙ-imidazolo, —(CHR)(CHR)ₙ-triazolo, —(CHR)(CHR)ₙ-pyrrolidino, optionally substituted by —(CH₂)ₙOH, —(CHR)(CHR)ₙ-3-hydroxy-pyrrolidino or —(CHR)(CHR)ₙ-piperidino, or
   —NR₂, —N(R)(CHR)ₙ-pyridino, —N(R)C(O)O-lower alkyl, —N(CH₂CF₃)C(O)O-lower alkyl, —N[C(O)O-lower alkyl]₂, —NR—NR—C(O)O-lower alkyl or —N(R)(CHR)ₙCF₃, —NRCF₃, —NRCF₂—R, —NRCF₂-lower alkenyl, —NRCHRF, —NRCHP-lower alkenyl;
   or is absent if X is —N═ or ═N—;
R⁵, R⁶ signify hydrogen, lower alkyl, lower alkoxy, amino, nitro, —SO₂NH₂ or halogen; or
R⁵ and R⁶ are interconnected to the group —O—CH₂—O—and form together with the C atoms to which they are attached an additional 5-membered ring;
R⁷, R⁸ signify hydrogen, lower alkyl, lower alkoxy, amino, nitro or halogen;
R⁹, R¹⁰ signify hydrogen or lower alkyl;
R¹¹, R¹² signifies hydrogen, lower alkyl, hydroxy, lower alkoxy, lower alkoxycarbonyloxy or lower alkanoyloxy;
R¹³, R¹⁴ signify hydrogen, tritium or lower alkyl;
R⁵, R¹⁶ signifies hydrogen, tritium, lower alkyl, hydroxy, lower alkoxy or are together an oxo group; or
X signifies —N═, ═N—, —N<, >C═ or ═C<;
Y signifies —N═, ═N—, —NH—, —CH═ or ═CH—; and
the dotted line may be a bond when R¹, R³ or R⁴ represent a bivalent atom, as well as with the pharmaceutically acceptable salts of each compound of the above formula and the racemic and optically active forms of each compound of the above formula.

mGluR5 antagonists are also described in WO 01/02342 and WO 01/02340. and have the following formulas, respectively: 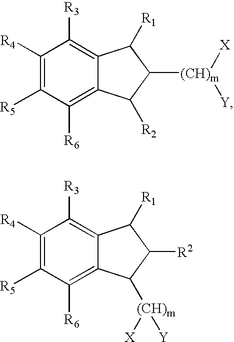
stereoisomers thereof, or pharmaceutically acceptable salts or hydrates thereof, wherein:
R1, and R2 are selected from the group comprising:
   1) H; or
   2) an acidic group selected from the group comprising carboxy, phosphono, phosphino, sulfono, sulfino, borono, tetrazol, isoxazol, —(CH₂)ₙ-carboxy, —(CH₂)ₙ-phosphono, —(CH₂)ₙ-phosphino, —(CH₂)ₙ-sulfono, —(CH₂)ₙ-sulfino, —(CH₂)ₙ-borono, —(CH₂)ₙ-tetrazol, and —(CH₂)ₙ-isoxazol, where n=1, 2, 3, 4, 5, or 6; or
X is an acidic group selected from the group comprising carboxy, phosphono, phosphino, sulfono, sulfino, borono, tetrazol, isoxazol;
Y is a basic group selected from the group comprising 1° amino, 2° amino, 3° amino, quaternary ammonium salts, aliphatic 1° amino, aliphatic 2° amino, aliphatic 3° amino, aliphatic quaternary ammonium salts, aromatic 1° amino, aromatic 2° amino, aromatic 3° amino, aromatic quaternary ammonium salts, imidazol, guanidino, boronoamino, allyl, urea, thiourea;
m is 0, 1;
R3, R4, R5, R6 are independently H, nitro, amino, halogen, tritium, trifluoromethyl, trifluoroacetyl, sulfo, carboxy, carbamoyl, sulfamoyl or acceptable esters thereof;
   or a salt thereof with a pharmaceutically acceptable acid or base.

Further classes of mGluR5 antagonists are described in WO 00/73283 and WO 99/26927. These compounds have the formula: R-[Linker]-Ar;
wherein R is an optionally substituted straight or branched chain alkyl, arylalkyl, cycloalkyl, or alkylcycloalkyl group preferably containing 5-12 carbon atoms. Ar is an optionally substituted aromatic, heteroaromatic, arylalkyl, or heteroaralkyl moiety containing up to 10 carbon atoms and up to 4 heteroatoms, and [linker] is —(CH₂)ₙ—, where n is 2-6, and wherein up to 4 CH₂ groups may independently be substituted with groups selected from the group consisting of C₁-C₃ alkyl, CHOH, CO, O, S, SO, SO₂, N, NH, and NO. Two heteroatoms in the [linker] may not be adjacent except when those atoms are both N (as in —N═N— of —NH—NH—) or are N and S as in a sulfonamide. Two adjacent CH₂ groups in [linker] also may be replaced by a substituted or unsubstituted alkene or alkyne group. Pharmaceutically acceptable salts of the compounds also are provided.

Another class of mGluR5 antagonists is described in WO 00/69816. These compounds have the formula: 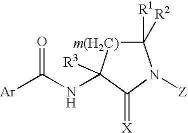
wherein,
n is O, 1 or 2;
X is O, S, NH, or NOH;
R¹ and R² are each independently H, CN, COOR, CONHR, C₁-C₆ alkyl, tetrazole, or R and R² together represent “═O”;
R is H or C₁-C₆ alkyl;
R³ is C₁-C₆ alkyl, C₂-C₆ alkenyl, C₃-C₆ cycloalkyl, —CH₂OH, —CH₂O-alkyl, —COOH;
Ar is an unsubstituted or substituted aromatic or heteroaromatic group;
Z represents a group of the formulae 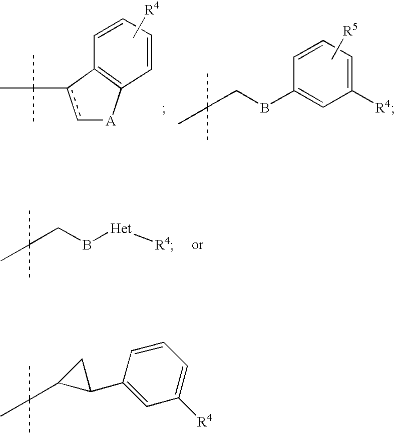
   wherein,
R⁴ and R⁵ are each independently H, halogen, C₁-C₆ alkoxy, —OAr, C₁-C₆ alkyl, —CF₃, COOR, CONHR, —CN, —OH, —COR, —S—(C₁-C₆ alkyl), —SO₂(C₁-C₆ alkyl);
A is CH₂, O, NH, NR, S, SO, SO₂, CH₂—CH₂, CH₂O, CHOH, C(O); wherein R is as defined above;
B is CHR, CR₂, C₁-C₆ alkyl, C(O), —CHOH, —CH₂—O, —CH═CH, CH₂—C(O), CH₂—S, CH₂—S(O), CH₂—SO₂; —CHCO₂R; or —CH—NR₂, wherein R is as defined above;
Het is a heterocycle such as furan, thiophene, or pyridine;
   or a pharmaceutically acceptable salt thereof.

mGluR1 antagonists are described in WO 00/26199 and WO 00/26198 and have the following formula: 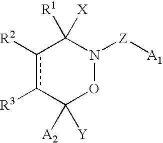
in which,
R¹, R² and R³ are independently hydrogen, (C₁-C₆)alkyl, (C₂-C₆)alkenyl, (C₃-C₁₀)cycloalkyl, unsubstituted or substituted aryl, unsubstituted or substituted aryl(C₁-C₆)alkyl, unsubstituted or substituted aryl(C₂-C₆)alkenyl, halo, carboxy, (C₁-C₆)alkoxycarbonyl or —(CH₂)ₘ—OH, wherein m is 1, 2 or 3; —
[symbol] indicates a single or a double bond;
X and Y are each independently hydrogen, or X and Y together represent a bridge of the formula —(CH₂)ₙ—, where n is 1 or 2;
A₁ and A₂ are each independently an unsubstituted or substituted aryl;
Z is —CO—, —SO₂— or —CH₂—; provided that, when Z is —CO—, A₁ is not 3,4,5-trimethoxyphenyl;
   or a pharmaceutically acceptable salt or ester thereof.

Another class of mGluR5 antagonists is described in WO 99/54280. These compounds have the formula: 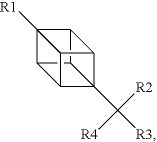
wherein,
R1 can be an acidic group selected from the group consisting of carboxyl, phosphono, phosphino, sulfono, sulfino, borono, tetrazol, isoxazol, —CH₂-carboxyl, —CH₂-phosphono, —CH₂-phosphino, —CH₂-sulfono, —CH₂-sulfino, —CH₂-borono, —CH₂-tetrazol, —CH₂-isoxazol and higher homologues thereof,
R2 can be a basic group selected from the group consisting of 1° amino, 2° amino, 3° amino, quaternary ammonium salts, aliphatic 1° amino, aliphatic 2° amino, aliphatic 3° amino, aliphatic quaternary ammonium salts, aromatic 1° amino, aromatic 2° amino, aromatic 3° amino, aromatic quaternary ammonium salts, imidazol, guanidino, boronoamino, allyl, urea, thiourea;
R3 can be H, aliphatic, aromatic or heterocyclic;
R4 can be an acidic group selected from the group consisting of carboxyl, phosphono, phosphino, sulfono, sulfino, borono, tetrazol, isoxazol; stereoisomers thereof;
   and pharmaceutically acceptable salts thereof.

mGluR5 antagonists are also described in WO 99/08678 and have the following formula: 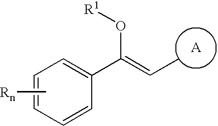
wherein R signifies halogen or lower alkyl;
n signifies 0-3;
R¹ signifies lower alkyl; cycloalkyl; benzyl optionally substituted by hydroxy, halogen, lower alkoxy or lower alkyl; benzoyl optionally substituted by amino, lower alkylamino or di-lower alkylamino; acetyl or cycloalkyl-carbonyl; and [symbol] signifies an aromatic 5-membered residue which is bonded via a N-atom and which contains further 1-3 N atoms in addition to the linking N atom,
   as well as their pharmaceutically acceptable salts.

Antagonists suitable for use with the invention can reduce activation of the mGluR by a ligand by, for example, at least about 10%, at least about 20%, at least about 30%, at least about 40%, at least about 50%, at least about 60%, at least about 70%, at least about 80%, at least about 90%, at least about 95%, or at least about 99% at a concentration of the antagonist. The percent antagonism represents the percent decrease in activity of mGluR, e.g., mGluR5, in a comparison of assays in the presence and absence of the antagonist.

In one embodiment, an antagonist for use in the invention may be a non-specific antagonist that is an antagonist of mGluR5 in general. In a specific embodiment, the Group I mGluR antagonist antagonizes mGluR5. In another embodiment, the Group I mGluR antagonist antagonizes. A selective antagonist is an antagonist that antagonizes either mGluR1 or mGluR5, but antagonizes other mGluR5 only weakly or substantially not at all, or at least antagonizes other mGluR5 with an IC₅₀ at least 10 or even 100 or 1000 times greater than the IC₅₀ at which it antagonizes mGluR5, mGluR1 or endocannabinoid recetors (e.g., CB1 receptors). The term “IC₅₀” means the concentration of a compound that inhibits an activity or property by 50%, e.g., by reducing the frequency of a cellular event, such as cell signaling or axon potential conduction, by 50%, by reducing binding of a competitor peptide to a mGluR or endocannabinoid receptor by 50% or by reducing the level of an activity, such as glutamate release from a presynaptic nerve terminal by 50%. Preferred antagonists are those which can selectively antagonize mGluR5 at low concentrations, for example, those that cause a level of antagonism of 50% or greater at a concentration of 100 μg/ml or less.

The Group I mGluR antagonist can be at least one member selected from the group consisting of (E)-6-methyl-2-styryl-pyridine (SIB 1893), 6-methyl-2 (phenylazo)-3-pyridinol, α-methyl-4-carboxyphenylglycine (MCPG), 2-methyl-6-(phenylethynyl)-pyridine (MPEP), (RS)-1-Aminoindan-1,5-dicarboxylic acid (AIDA), DL-2-Amino-3-phosphonopropionic acid (DL-AP3), (S)-4-Carboxyphenylglycine ((S)-4C3HPG), 7-(Hydroxyimino)cyclopropa[b]chromen-1a-carboxylate ethyl easter (CPCCOEt), α-Amino-4-hexyl-2,3-dihydro-3-oxo-5-isoxazolepropanoic acid (HexylHIBO), (αS)-a-Amino-4-hexyl-2,3-dihydro-3-oxo-5-isoxazolepropanoic acid ((S)-HexylHIBO), (S)-(+)-α-Amino-4-carboxy-2-methylbenzeneacetic acid (LY 367385), N-Phenyl-7-(hydroxyimino)cyclopropa[b]chromen-1a-carboxamide (PHCCC), and 6-Methyl-2-(phenylazo)-3-pyridinol (SIB 1757).

In another embodiment, the invention is a method of treating a subject comprising the step of administering at least one compound that down regulates endocannabinoid signaling (e.g., antagonists of CB-1 receptors).

“Down regulates endocannabinoid signaling,” as used herein, refers to any decrease or any inhibition in a cellular process or a cellular event or intermediate in a cellular event associated with any mechanism whereby endocannabinoid receptors mediate a biological response, in particular, responses that involve CB1 receptors. For example, down regulation can be the prevention or any decrease in binding of a signal external to a cell (a first messenger), such as a ligand (e.g., an endocannobinoid), to a receptor (e.g., presynpatic CB1 receptors). Down regulation can be disruption of a cellular process following binding of an external signal (e.g., ligand) to an endocannabinoid receptor such as the prevention of activation of adenylyl cyclase, phospholipase C (PLC) or phospholipase D (PLD). Down regulation can also be disruption of a cellular process following binding of an external signal to an ednocannabinoid receptor such as the prevention of activation of a G-protein (Gs, Gq), a decrease in a G-protein (Gs, Gq) activation, prevention of activation of second messengers activated by endocannabinoids (e.g., cAMP, IP₃, diacylglycerol (DAG), PLC, PLD) or a decrease in the activity of an intracellular effector, such as a cAMP-dependent protein kinase, protein kinase C (PKC) or calcium release. FIG. 3 depicts down regulation of endocannabinoid signaling and thereby treatment of OCD.

Compounds used in the methods of the invention that down regulate mGluR signaling or down regulate endocannabinoid signaling can interfere with binding of a ligand to a mGluR or an endocannabinoid recptor (e.g., CB1 receptor). “Interfere,” as used herein when referring to binding of a ligand to a receptor, means any effect on ligand-receptor binding that occurs in the presence of the compound that prevents the ligand from activating the receptor or mediating cell signaling.

Compounds used in the methods of the invention that down regulate mGluR signaling or down regulate endocannabinoid signaling can alter processes in cell signaling pathways, such as cAMP-dependent protein kinase. “Alter,” as used herein in reference to cell signaling pathways, means any difference in the cell signaling pathway, molecule or intermediate in the cell signaling pathway that occurs in the presence of the compound. An alteration can be, for example, a structural change in a protein kinase that inhibits the activity of the kinase.

Compounds that down regulate endocannabinoid signaling can be antagonists of endocannibinoid receptors. In a particular embodiment, the endocannabionoid antagonist is a CB-2 receptor antagonist. Suitable CB-1 antagonists are, for example, N-(Piperidin-1-yl)-5-(4-iodophenyl)-1-(2,4-dichlorophenyl)-4-methyl-1H-pyrazole-3-carboxamide (AM251), 1-(2,4-Dichlorophenyl)-5-(4-iodophenyl)-4-methyl-N-4-morpholinyl-1H-pyrazole-3-carboxamide (AM281), and 5-(4-Chlorophenyl)-1-(2,4-dichlorophenyl)-4-methyl-N-(piperidin-1-yl)-1H-pyrazole-3-carboxamide (SR141716A).

An antagonist of an endocannabinoid receptor is a substance that diminishes or abolishes the effect of a ligand (an endocannabinoid or agonist) that activates an endocannabinoid receptor. The endocannabinoid antagonist can be, for example, similar to the Group I mGluR antagonist, a chemical antagonist, a pharmacokinetic antagonist, an antagonist by receptor block, a non-competitive antagonist, or a physiological antagonist as described above for a Group I mGluR antagonist; however, the antagonist acts at the level of an endocannabinoid receptor rather than a Group I mGluR antagonist.

An “effective amount,” also referred to herein as a “therapeutically effective amount,” when referring to the amount of a compound (e.g., drug) or composition (e.g., pharmaceutical composition containing a drug) that down regulates mGluR signaling (e.g., Group I mGluR signaling), is defined as that amount, or dose, of a compound or composition that, when administered to a subject having OCD, is sufficient for therapeutic efficacy (e.g., an amount sufficient to reduce obsessions, such as contamination fears of germs or dirt, imagining having harmed self or others, imagining losing control, imagining aggressive urges, intrusive sexual thoughts or urges, excessive religious or moral doubts, forbidden thoughts, a need to have things in particular manner, a need to tell, ask or confess; and/or compulsions, such as washing of hands, repeating tasks, checking, touching, counting, ordering, arranging, hoarding or saving and praying) in a subject with OCD.

The methods of the present invention can be accomplished, for example, by the administration of a compound that down regulates Group I mGluR signaling (e.g., antagonists of Group I mGluR) or down regulates endocannabinoid receptor signaling (e.g., CB1 receptor signaling) by enteral or parenteral means. Specifically, the route of administration is by oral ingestion (e.g., tablet, capsule form). Other routes of administration as also encompassed by the present invention including intramuscular, intravenous, intraarterial, intraperitoneal, or subcutaneous routes and nasal administration. Suppositories or transdermal patches can also be employed.

One or more compounds that down regulate Group I mGluR signaling or endocannabinoid signaling can be administered to the subject. For example, the subject can be treated with a compound that down regulates Group I mGluR signaling by preventing binding of a ligand to a Group I mGluR or a compound that disrupts intracellular signaling following binding of a ligand to a Group I mGluR. Likewise, a compound that down regulates endocannbinoid signaling can prevent binding of an endocannabinoid to a CB1 receptor or disrupt cell signaling following endocannabinoid binding to a CB1 receptor.

Compounds that down regulate Group I mGluR signaling or endocannabinoid signaling can be co-administered. Coadminstration can include simultaneous or sequential administration of the compounds that down regulate Group I mGluR signaling and/or compounds that down regulate endocannabinoid signaling.

Compounds that down regulate Group I mGluR signaling or endocannabinoid signaling can be administered alone or can be coadministered to the subject with another treatment regimen, such as SRIs, SSRIs or anxiolytics.

Compounds that down regulate Group I mGluR signaling or endocannabinoid signaling can be administered alone or as admixtures with conventional excipients, for example, pharmaceutically, or physiologically, acceptable organic, or inorganic carrier substances suitable for enteral or parenteral application which do not deleteriously react with the compounds. Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the compound that down regulates mGluR signaling. The preparations can also be combined, when desired, with other active substances to reduce metabolic degradation. The compound that down regulates Group I mGluR signaling can be administered in a single or in more than one dose over a period of time to confer the desired effect (e.g., alleviate urges and repetitive actions).

When parenteral application is needed or desired, particularly suitable admixtures for a compound that down regulates Group I mGluR signaling or endocannabinoid are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compound that down regulates mGluR signaling can also be incorporated into liposomes or administered by transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309 the teachings of which are hereby incorporated by reference.

The compounds (e.g., Group I mGluR antagonists, Group II mGluR agonist, Group III mGluR agonist, compounds that down regulate endocannabinoid signaling) employed in the methods of the invention can be administered in a dose of between about 0.1 mg/kg to about 1 mg/kg body weight; about 1 mg/kg to about 5 mg/kg body weight; between about 5 mg/kg to about 15 mg/kg body weight; between about 10 mg/kg to about 25 mg/kg body weight; between about 25 mg/kg to about 50 mg/kg body weight; or between about 50 mg/kg body weight to about 100 mg/kg body weight. The compounds can be administered in doses of about 0.01 mg, about 0.1 mg, about 1 mg, about 2 mg, about 10 mg, about 25 mg, about 50 mg, 100 mg, about 200 mg, about 250 mg, about 300 mg, about 350 mg, about 400 mg, about 500 mg, about 600 mg, about 700 mg, about 900 mg, about 1000 mg, about 1200 mg, about 1400 mg, about 1600 mg or about 2000 mg. The compounds can be administered once a day or multiple (e.g., two, three, four, five) times per day.

The dosage and frequency (single or multiple doses) administered to a subject can vary depending upon a variety of factors, including the duration of OCD and obsessions and compulsions associated with OCD, whether the subject suffers from other disorders, conditions or syndromes, age, sex, health, body weight and body mass index of the subject; nature and extent of symptoms or conditions of OCD and other conditions or symptoms of the subject (e.g., fragile X syndrome), kind of concurrent treatment (e.g., SRI, SSRI, anxiolytics), or other health-related problems. Other therapeutic regimens or agents can be used in conjunction with the methods of the present invention. For example, the administration of the compound that down regulates Group I mGluR signaling can be accompanied by antidepressant (e.g., SRI, SSRI) and/or anxiolytic treatment. Adjustment and manipulation of established dosages (e.g., frequency and duration) are well within the ability of those skilled in the art.

Although not to be considered limiting in any way, Group I mGluR signaling is believed to have many effects, some of the mechanisms of Group I mGluR signaling and known or theorized effects, particularly with respect to FMRP and fragile X syndrome, are described below. The methods described herein may be used to treat at least one of the effects described below in subjects with, for example, at least one condition selected from the group consisting of fragile X syndrome, autism and mental retardation.

Group I Metabotropic Glutamate Receptors

Group 1 metabotropic glutamate receptor (Gp1 mGluR) activation is exaggerated in the absence of the fragile X mental retardation protein (FMRP) and may reflect altered dendritic protein synthesis. Abnormal mGluR signaling can be responsible for diverse psychiatric and neurological symptoms in fragile X syndrome, including delayed cognitive development, seizures, anxiety, movement disorders and obesity.

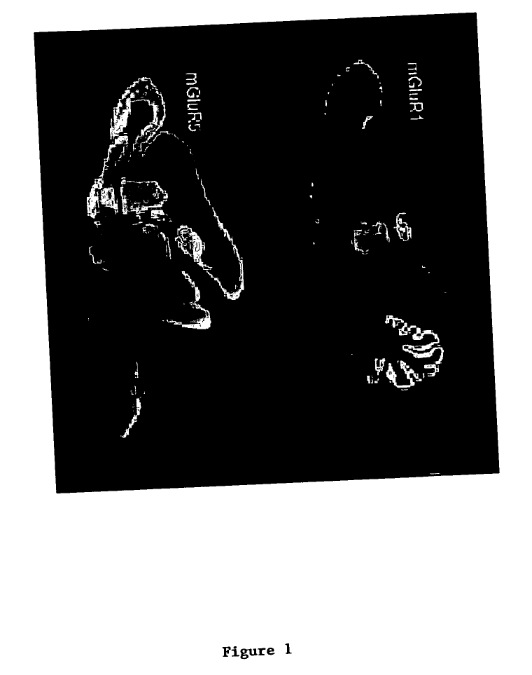

Polyribosomes and the molecular machinery of protein synthesis are localized near dendritic spines, major sites of excitatory synaptic transmission and plasticity (Steward & Schuman 2001). Glutamate is the neurotransmitter at most excitatory synapses in the brain, and Group 1 (Gp 1) metabotropic glutamate receptors (mGluR1 and mGluR5) can be potent stimuli for protein synthesis (Job & Eberwine 2001, Shin et al 2004, Todd et al 2003, Weiler & Greenough 1993). In certain cases, many of the lasting functional consequences of Gp1 mGluR activation have been found to be dependent on mRNA translation, but not transcription (Huber et al 2000, Karachot et al 2001, Lee et al 2002, Merlin et al 1998, Raymond et al 2000, Snyder et al 2001, Stoop et al 2003, Vanderklish & Edelman 2002, Zho et al 2002). Other than the common requirement for protein synthesis, the precise consequence of activating Gp I mGluR5 varies widely, depending on the neuron and the circuit in which it resides (FIG. 1). Systemic activation or inhibition of Gp I mGluR-mediated protein synthesis by genetic or pharmacological means can have diverse effects.

The fragile X mental retardation protein (FMRP) may regulate dendritic protein synthesis. However, recent biochemical and cell-biological studies regarding the role of FMRP in dendritic protein synthesis is confusing and, at times, contradictory. FMRP is contained within ribonucleoprotein granules that traffic specific mRNAs (including Fmrl) to sites of synaptic transmission. Activation of Gp1 mGluR5 on cultured hippocampal neurons with the selective agonist DHPG ((R,S)-3,5-dihydroxyphenylglycine) triggers the delivery of FMRP to dendrites (Antar et al 2004). In cultured cortical neurons, expression of both FMRP and the synaptic protein PSD-95 is increased after activating Gp 1 mGluR5. However, DHPG treatment fails to increase PSD-95 levels in cultures prepared from Fmr1 knockout (KO) mice lacking FMRP (Todd et al 2003). Similarly, DHPG fails to stimulate polyribosome assembly in synaptosomes prepared from the cortex of Fmr1 KO mice (Weiler et al 2004). Together, these data are consistent with the proposal that FMRP is a requirement for dendritic protein synthesis. On the other hand, there is also data showing that synthesis of some proteins (e.g., MAP1b) is repressed by FMRP and that this repression is relieved in the Fmr1 KO and in humans with fragile X (reviewed by (Bear et al 2004); see also Warren article, this issue). The disparate findings may be related to differences in the tissue, the preparation, the mRNA, and the subcellular compartment under investigation (Miyashiro & Eberwine 2004).

The functional consequence of Gp1 mGluR activation differs in the Fmr1 knock out (KO) mouse. The physiological response in a form of synaptic plasticity, long-term depression (LTD), is triggered in the CA1 region of hippocampus by appropriate stimulation of mGluR5. This type of LTD is protein synthesis-dependent (Huber et al 2000) and expressed, in part, by internalization of glutamate receptors (Snyder et al 2001). In the Fmr1 KO mice, the mGluR-induced LTD was increased in the hippocampus (Huber et al 2002), consistent with the hypothesis that FMRP normally represses the protein synthesis required for stable expression of mGluR-dependent LTD. In the absence of repression, an increase in LTD was observed in the FMRP KO mice. Regardless of the specific mechanism involved, the data showed that one functional consequence of mGluR activation is exaggerated in the absence of the fragile X protein.

It was unclear whether other functional consequences of mGluR-dependent protein synthesis are exaggerated and, if so, whether this could account for aspects of the phenotype in fragile X syndrome. The “mGluR theory” of fragile X syndrome was proposed (Bear et al 2004). The theory is based on two assumptions: (1) that many lasting consequences of Gp1 mGluR activation require protein synthesis, and (2) that these are exaggerated in the absence of FMRP. Symptoms of fragile X syndrome or conditions associated with fragile X syndrome may arise from excessive signaling through Gp1 mGluR5, which may be inhibited by treatment with compounds that inhibit the receptors and/or the downstream intracellular signals that Gp1 mGluR5 initiate.

Cognitive Development

Fragile X syndrome is characterized by moderate to severe mental retardation (Bakker & Oostra 2003, Hagerman 2002, Hagerman & Hagerman 2002). Cognition is an emergent property of the cerebral cortex, and the trajectory of cognitive development depends on experience-dependent modifications of synaptic connections among cortical neurons. Synaptic excitation in the cortex is mediated by AMPA and NMDA receptors (the major classes of glutamate-gated ion channel). A biochemical phenotype in the Fmr1 KO mouse is reduced expression of the AMPA receptor subunit protein GluR1 in synaptic plasma membranes prepared from frontal cortex (Li et al 2002).

The experience-dependent delivery and removal of AMPA receptors from cortical synapses is essential for normal cortical developments as well as for adult learning and memory. A great deal has been learned about the mechanisms responsible for this synaptic plasticity by the study of the experimental phenomena of long-term potentiation (LTP) and long-term depression (Malenka & Bear 2004). One form of LTD is induced in the CA1 region of hippocampus by activation of mGluR5 (Huber et al 2001), the major Gp1 mGluR in the cerebral cortex (FIG. 1). In cultured hippocampal neurons, activation of mOluR5 with DHPG stimulates the loss of AMPA receptors from synapses, and this is believed to model a mechanism used during cortical development to refine synaptic connections (Snyder et al 2001). Both LTD and the internalization of AMPA receptors are protein synthesis-dependent, and both responses are increased in neurons from the Fmr1 KO mouse (Huber et al 2002). Excessive mGluR-dependent LTD during development may explain the loss of AMPA receptor protein in synaptic plasma membranes from the KO mice.

Neurons in the cerebral cortex of mice and humans lacking FMRP also have a greater proportion of long, thin dendritic spines (Hinton et al 1991, Irwin et al 2001, Rudelli et al 1985). Spine abnormalities have long been associated with human mental retardation of unknown etiology (Purpura 1974), as well as with Down's and Rett syndromes (Kaufmann & Moser 2000). This phenotype may also be related to excessive Gp1 mGluR signaling. Vanderklish and Edelman (Proc. Natl. Acad. Sci USA, 99:1639-1644 (2002)) described prolonged treatment of hippocampal neurons with DHPG increased the proportion of long, thin dendritic spines (Vanderklish & Edelman 2002). These structural changes and LTD are may be related, because synapses on thin spines have a smaller postsynaptic density, fewer AMPA receptors, and a reduced number of synaptic vesicles docked at the presynaptic active zone (Harris & Stevens 1989, Nusser et al 1998, Schikorski & Stevens 1997).

Exaggerated mGluR5 signaling may contribute to the altered trajectory of cortical development in fragile X syndrome. A number of genetic tests are feasible in mice; for example, by crossing Fmr1 KO mice with animals deficient in mGluR5. Chronic treatment with an mGluR5 antagonist during a critical period of postnatal development may be “disease modifying” in animals and humans lacking FMRP.

In the Drosophila model for fragile X syndrome, flies lack dfmr1, the homologue of FMR1 in humans, display altered courtship behavior, decreased memory in a conditioned courtship assay, and alterations in the structure of the brain (the mushroom bodies) (McBride et al 2005). McBride, Jongens and colleagues have found that all of these phenotypes in mutant flies are rescued if they are raised with food containing MPEP, or several other drugs that are predicted to affect signaling by the Drosophila mGluR, DmGluRA. The mushroom body defect could only be rescued when drug treatment began at the larval stage of development, but significant behavioral rescue occurred even when treatment began in adult flies (McBride et al 2005). One of the effective agents in flies was lithium, currently in widespread use in humans for treatment of mood disorders.

Seizure Disorder

A large proportion of humans with fragile X syndrome have seizures during childhood (Hagerman 1987, Hagerman 2002, Hagerman & Hagerman 2002), and a robust phenotype in the Fmr1 KO mice is audiogenic seizures. There are connections between excessive Gp1 mGluR activation and epilepsy (Wong et al 2002).

Electroencephalographic measurements reveal two types of synchronized discharge in epilepsy: brief interictal sharp waves with no perceptible behavioral correlate, and prolonged ictal bursts, lasting from seconds to minutes, that produce seizures (Zifkin & Cracco 1990). Hippocampal area CA3 has been used to model the mechanisms involved. Bathing a hippocampal slice in drugs that block inhibition leads to the generation of regularly spaced bursts of synchronous activity in CA3 pyramidal cells that resemble interictal sharp waves. These brief bursts will continue for hours in vitro without evolving to ictal-like activity. However, ictal-like activity rapidly appears and persists following transient activation of Gp1 mGluRs (Merlin et al 1998). The requirements for this lasting consequence of mGluR activation are strikingly similar to those for LTD. Induction of ictal-like activity requires activation of extracellular signal-regulated kinase (ERK), a subclass of the mitogen-activated protein kinases (Zhao et al 2004), and mRNA translation, but not transcription (Merlin et al 1998).

Protein synthesis-dependent response to mGluR activation, like LTD, may be exaggerated in the absence of FMRP. Ictal-like activity emerges spontaneously in slices from the Fmr1 KO mouse, and that this could be reversed by administering the mGluR5 antagonist MPEP (S. Chuang, Q. Yan, R. P. Bauchwitz, R. K. S. Wong. Program No. 228.5. 2004 Abstract Viewer/Itinerary Planner. Washington, D.C.: Society for Neuroscience, 2004. Online). Thus, in slices from the mutant (but not wild-type), the endogenous activation of mGluR5 by synaptically released glutamate was sufficient to trigger the protein synthesis required for establishment of ictal-like epileptiform activity.

Antagonists of mGluR5 have previously been shown to have broad anticonvulsant actions (Spooren et al 2001).

Anxiety Disorder

Sensory hyperarousal and anxiety are the sine qua non of fragile X syndrome in humans (Hagerman & Hagerman 2002). The biological bases of anxiety disorders are poorly understood, but much attention is focused on the control of the hypothalamic-pituitary-adrenal axis by the amygdala. The amygdala is critical for the expression of learned fear. For example, repeated pairing of an auditory stimulus (a tone) with a footshock causes the animal to exhibit fear in response to the tone alone. There is evidence that the tone-shock pairing induces LTP of the synapses bringing the auditory information to the lateral amygdala (Maren & Quirk 2004). LTP in the lateral amygdala requires activation of mGluR5 (Rodrigues et al 2002, Rodrigues et al 2004). LTP may be dependent on translation of preexisting mRNA, and may be enhanced in the Fmr1 KO mouse.

mGluR5 antagonists may be effective anxiolytics. mGluR5 antagonists exhibit the widest and most robust anxiolytic activity in preclinical models seen to date (Spooren & Gasparini 2004). The effects are comparable to those of benzodiazepines with less sedative activity. Thus, although the site(s) and mechanism(s) of action remain to be determined, mGluR5 antagonists may have therapeutic potential for anxiety in fragile X syndrome.

Disorders of Movement

Two disorders of movement are common in fragile X syndrome: incoordinated voluntary movements and repetitive, obsessive-compulsive-like behaviors (Hagerman & Hagerman 2002). Gp1 mGluR5 are highly expressed in two motor-control structures that might contribute to these symptoms: the cerebellum and the striatum (FIG. 1).

Theories of cerebellar function suggest that motor learning occurs by adjustments of the strength of parallel fiber synapses onto Purkinje neurons, based on the relative timing of the parallel fiber activity and “error signals” conveyed by the climbing fibers arising from the inferior olive. Coincident activation of parallel and climbing fibers induces LTD at the parallel fiber-Purkinje cell synapse (Bear & Linden 2001, Ito 1989). Climbing fiber activation is permissive for LTD by elevating intracellular calcium ion concentration; however, the signal that marks the parallel fiber synapse for depression is local activation of Gp1 mGluRs. In the case of cerebellar LTD, the critical receptor is mGluR1, rather than mGluR5. Similar to the situation in the hippocampus, mGluR-dependent LTD in the cerebellum requires activation of ERK (Endo & Launey 2003), the translation of preexisting mRNA (Karachot et al 2001), and is expressed as a loss of AMPA receptors (Steinberg et al 2004). Cerebellar LTD was examined in the Fmr1 KO mouse and found to be increased, consistent with the predictions of the mGluR theory. This change in cerebellar synaptic plasticity correlated with impairments in motor learning as assessed by associative eyeblink conditioning. Moreover, comparable defects in eyeblink conditioning were observed in humans with fragile X syndrome. These results suggest that dampening signaling through mGluR1 also may be beneficial in treating fragile X syndrome.

Gp1 mGluRs also play a central role in synaptic plasticity in the striatum believed to be important for development of habitual motor routines (Gerdeman et al 2003, Gubellini et al 2004). High-frequency stimulation of the cortical afferents to striatal medium spiny neurons can elicit either LTP or LTD, depending on a number of variables such as age and position within the striatum. Both forms of synaptic plasticity require activation of mGluR1 and/or mGluR5; LTP requires, in addition, activation of NMDA receptors. At present, the picture is most clear for LTD in the dorsal-lateral striatum. Similar to the parallel fiber-Purkinje cell synapse, LTD is induced at corticostriatal synapses by the simultaneous activation of Gp1 mGluRs and a rise in postsynaptic calcium entering through voltage gated channels. However, unlike the cerebellum, induction of striatal LTD also requires dopamine signaling, and LTD is expressed presynaptically as a reduced probability of glutamate release. The retrograde messenger, signalling from postsynaptic mGluRs to the presynaptic axon terminal, is an endocannabinoid acting on presynaptic CB1 receptors (Gerdeman et al 2002). A role for translation of preexisting mRNA following mGluR activation has not yet been examined.

Excessive Gp1 mGluR-dependent LTD may occur in the striatum of the Fmr1 KO mice. The development of stereotypies may be a consequence of LTD-like changes in the dorsolateral striatum (Graybiel et al 2000). Striatal activation is deficient in humans with obsessive-compulsive disorder (Graybiel & Rauch 2000, Rauch et al 1997). Antagonists of Gp 1 mGluRs (mGluR5, in particular) may be beneficial for the treatment of compulsive motor routines in fragile X syndrome.

Other Symptoms

Other symptoms associated with fragile X syndrome include obesity, irritable bowel, and hyperalgesia. The neurobiological basis for these symptoms remains to be determined in fragile X syndrome.

Obesity may arise from altered signaling in the hypothalamus. The ventromedial hypothalamus responds to hormones that signal energy demand and incites feeding behavior by connections with the lateral hypothalamus (Saper et al 2002). Both the ventromedial and lateral hypothalamus have high levels of mGluR5 expression (van den Pol et al 1995). Very recently, it was reported that mGluR5 knockout mice have diminished appetite and weigh less than wildtype littermates. Moreover, treatment of rats with an mGluR5-selective antagonist decreased food intake and caused weight loss (Bradbury et al 2004). Exaggerated mGluR5 signaling may be responsible for obesity in fragile X syndrome.

Gut motility is controlled by a complex interaction of the enteric and central nervous systems (Hunt & Tougas 2002). A population of secretomotor neurons in the ileum contain mGluR5 (Liu & Kirchgessner 2000). Local application of mGluR5 agonists and antagonists increase and decrease, respectively, gut motility (Hu et al 1999).

A majority of patients with irritable bowel syndrome also have altered pain perception (Hunt & Tougas 2002), and hyperalgesia is a common complaint in fragile X syndrome. mGluR5 is expressed by nociceptive C fibers, where it has been implicated in the mechanisms of hyperalgesia.

### REFERENCES

Antar L N, Afroz R, Dictenberg J B, Carroll R C, Bassell G J. 2004. Metabotropic glutamate receptor activation regulates fragile X mental retardation protein and FMR1 mRNA localization differentially in dendrites and at synapses. J Neurosci 24: 2648-55
Bakker C E, Oostra B A. 2003. Understanding fragile X syndrome: insights from animal models. Cytogenet Genome Res 100: 111-23
Bear M F, Huber K M, Warren S T. 2004. The mGluR theory of fragile X mental retardation. Trends Neurosci 27: 370-7
Bear M F, Linden D J. 2001. The mechanisms and meaning of long-term synaptic depression in the mammalian brain. In Synapses, ed. W M Cowan, T C Sudhoff, C F Stevens, pp. 455-517. Baltimore: The Johns Hopkins University Press
Bradbury M J, Campbell U, Giracello D, Chapman D, King C D, et al. 2004. Metabotropic glutamate receptor, mGlu5, is a mediator of appetite and energy balance in rats and mice. J Pharmacol Exp Ther
Chiamulera C, Epping-Jordan M P, Zocchi A, Marcon C, Cottiny C, et al. 2001. Reinforcing and locomotor stimulant effects of cocaine are absent in mGluR5 null mutant mice. Nat Neurosci 4: 873-4
Endo S, Launey T. 2003. ERKs regulate PKC-dependent synaptic depression and declustering of glutamate receptors in cerebellar Purkinje cells. Neuropharmacology 45: 863-72
Gerdeman G L, Partridge J G, Lupica C R, Lovinger D M. 2003. It could be habit forming: drugs of abuse and striatal synaptic plasticity. Trends Neurosci 26: 184-92
Gerdeman G L, Ronesi J, Lovinger D M. 2002. Postsynaptic endocannabinoid release is critical to long-term depression in the striatum. Nat Neurosci 5: 446-51
Graybiel A M, Canales J J, Capper-Loup C. 2000. Levodopa-induced dyskinesias and dopamine-dependent stereotypies: a new hypothesis. Trends Neurosci 23: S71-7
Graybiel A M, Rauch S L. 2000. Toward a neurobiology of obsessive-compulsive disorder. Neuron 28: 343-7
Gubellini P, Pisani A, Centonze D, Bemardi G, Calabresi P. 2004. Metabotropic glutamate receptors and striatal synaptic plasticity: implications for neurological diseases. Prog Neurobiol 74: 271-300
Hagerman R J. 1987. Fragile X syndrome. Curr Probl Pediatr 17: 621-74
Hagerman R J. 2002. The physical and behavioral phenotype. In Fragile X Syndrome: Diagnosis, Treatment, and Research, ed. R J Hagerman, P Hagerman, pp. 3-109. Baltimore: The Johns Hopkins University Press
Hagerman R J, Hagerman P J, eds. 2002. Fragile X Syndrome. Baltimore: The Johns Hopkins University Press. 540 pp.
Harris K M, Stevens J K. 1989. Dendritic spines of CA 1 pyramidal cells in the rat hippocampus: serial electron microscopy with reference to their biophysical characteristics. J Neurosci 9: 2982-97
Hinton V J, Brown W T, Wisniewski K, Rudelli R D. 1991. Analysis of neocortex in three males with the fragile X syndrome. Am J Med Genet 41: 289-94
Hu H Z, Ren J, Liu S, Gao C, Xia Y, Wood J D. 1999. Functional group I metabotropic glutamate receptors in submucous plexus of guinea-pig ileum. Br J Pharmacol 128: 1631-5
Huber K M, Gallagher S M, Warren S T, Bear M F. 2002. Altered synaptic plasticity in a mouse model of fragile X mental retardation. Proc Natl Acad Sci USA 99: 7746-50
Huber K M, Kayser M S, Bear M F. 2000. Role for rapid dendritic protein synthesis in hippocampal mGluR-dependent long-term depression. Science 288: 1254-7
Huber K M, Roder J C, Bear M F. 2001. Chemical induction of mGluR5- and protein synthesis—dependent long-term depression in hippocampal area CA1. J Neurophysiol 86: 321-5
Hunt R H, Tougas G. 2002. Evolving concepts in functional gastrointestinal disorders: promising directions for novel pharmaceutical treatments. Best Pract Res Clin Gastroenterol 16: 869-83
Irwin S A, Patel B, Idupulapati M, Harris J B, Crisostomo R A, et al. 2001. Abnormal dendritic spine characteristics in the temporal and visual cortices of patients with fragile-X syndrome: a quantitative examination. Am J Med Genet 98: 161-7
Ito M. 1989. Long Term Depression. In Annual Review of Neuroscience, ed. M W Cowan, E M Shooter, C F Stevens, R F Thompson, pp. 85-102. Palo Alto, Calif.: Annual Reviews, Inc.
Job C, Eberwine J. 2001. Identification of sites for exponential translation in living dendrites. Proc Natl Acad Sci USA 98: 13037-42
Karachot L, Shirai Y, Vigot R, Yamamori T, Ito M. 2001. Induction of long-term depression in cerebellar Purkinje cells requires a rapidly turned over protein. J Neurophysiol 86: 280-9
Kaufmann W E, Moser H W. 2000. Dendritic anomalies in disorders associated with mental retardation. Cereb Cortex 10: 981-91
Lee A C, Wong R K, Chuang S C, Shin H S, Bianchi R. 2002. Role of synaptic metabotropic glutamate receptors in epileptiform discharges in hippocampal slices. J Neurophysiol 88: 1625-33
Lee B, Platt D M, Rowlett J K, Adewale A S, Spealman R. 2004. Attenuation of behavioral effects of cocaine by the metabotropic glutamate receptor 5 antagonist 2-methyl-6-(phenylethynyl)-pyridine in squirrel monkeys: Comparison with dizocilpine. J Pharmacol Exp Ther
Li J, Pelletier M R, Perez Velazquez J L, Carlen P L. 2002. Reduced cortical synaptic plasticity and GluR1 expression associated with fragile X mental retardation protein deficiency. Mol Cell Neurosci 19: 138-51
Liu M, Kirchgessner A L. 2000. Agonist- and reflex-evoked internalization of metabotropic glutamate receptor 5 in enteric neurons. J Neurosci 20: 3200-5
Malenka R C, Bear M F. 2004. LTP and LTD: an embarrassment of riches. Neuron 44: 5-21
Maren S, Quirk G J. 2004. Neuronal signalling of fear memory. Nat Rev Neurosci 5: 844-52
McBride S M J, Choi C H, Wang Y, Liebelt D, Braunstein E, et al. 2005. Pharmacological rescue of synaptic plasticity, courtship behavior and mushroom body defects in a Drosophila model of Fragile X Syndrome. Neuron
Merlin L R, Bergold P J, Wong R K. 1998. Requirement of protein synthesis for group I mGluR-mediated induction of epileptiform discharges. J Neurophysiol 80: 989-93
Miyashiro K, Eberwine J. 2004. Fragile X syndrome: (What's) lost in translation? Proc Natl Acad Sci USA 101: 17329-30
Nusser Z, Lujan R, Laube G, Roberts J D, Molnar E, Somogyi P. 1998. Cell type and pathway dependence of synaptic AMPA receptor number and variability in the hippocampus. Neuron 21: 545-59
Purpura D P. 1974. Dendritic spine “dysgenesis” and mental retardation. Science 186: 1126-8
Rauch S L, Savage C R, Alpert N M, Dougherty D, Kendrick A, et al. 1997. Probing striatal function in obsessive-compulsive disorder: a PET study of implicit sequence learning. J Neuropsychiatry Clin Neurosci 9: 568-73
Raymond C R, Thompson V L, Tate W P, Abraham W C. 2000. Metabotropic glutamate receptors trigger homosynaptic protein synthesis to prolong LTP. J. Neurosci. 20: 969-76
Rodrigues S M, Bauer E P, Farb C R, Schafe G E, LeDoux J E. 2002. The group I metabotropic glutamate receptor mGluR5 is required for fear memory formation and long-term potentiation in the lateral amygdala. J Neurosci 22: 5219-29
Rodrigues S M, Schafe G E, LeDoux J E. 2004. Molecular mechanisms underlying emotional learning and memory in the lateral amygdala. Neuron 44: 75-91
Rudelli R D, Brown W T, Wisniewski K, Jenkins E C, Laure-Kamionowska M, et al. 1985. Adult fragile X syndrome. Clinico-neuropathologic findings. Acta Neuropathol (Berl) 67: 289-95
Saper C B, Chou T C, Elmquist J K. 2002. The need to feed: homeostatic and hedonic control of eating. Neuron 36: 199-211
Schikorski T, Stevens C F. 1997. Quantitative ultrastructural analysis of hippocampal excitatory synapses. J Neurosci 17: 5858-67
Shigemoto R, Mizuno N. 2000. Metabotropic glutamate receptors—immunocytochemical and in situ hybridization analysis. In Handbook of Chemical Neuroanatomy, ed. O P Ottersen, J Storm-Mathisen, pp. 63-98: Elsevier
Shin C Y, Kundel M, Wells D G. 2004. Rapid, activity-induced increase in tissue plasminogen activator is mediated by metabotropic glutamate receptor-dependent mRNA translation. J Neurosci 24: 9425-33
Snyder E M, Philpot B D, Huber K M, Dong X, Fallon J R, Bear M F. 2001. Internalization of ionotropic glutamate receptors in response to mGluR activation. Nat Neurosci 4: 1079-85
Spooren W, Gasparini F. 2004. mGlu5 receptor antagonists: a novel class of anxiolytics? Drug News Perspect 17: 251-7
Spooren W P, Gasparini F, Salt T E, Kuhn R. 2001. Novel allosteric antagonists shed light on mglu(5) receptors and CNS disorders. Trends Pharmacol Sci 22: 331-7
Steinberg J P, Huganir R L, Linden D J. 2004. N-ethylmaleimide-sensitive factor is required for the synaptic incorporation and removal of AMPA receptors during cerebellar long-term depression. Proc Natl Acad Sci USA
Steward O, Schuman E M. 2001. Protein synthesis at synaptic sites on dendrites. Annu Rev Neurosci 24: 299-325
Stoop R, Conquet F, Zuber B, Voronin L L, Pralong E. 2003. Activation of metabotropic glutamate 5 and NMDA receptors underlies the induction of persistent bursting and associated long-lasting changes in CA3 recurrent connections. J Neurosci 23: 5634-44
Todd P K, Mack K J, Malter J S. 2003. The fragile X mental retardation protein is required for type-I metabotropic glutamate receptor-dependent translation of PSD-95. Proc Natl Acad Sci USA 100: 14374-8
van den Pol A N, Romano C, Ghosh P. 1995. Metabotropic glutamate receptor mGluR5 subcellular distribution and developmental expression in hypothalamus. J Comp Neurol 362: 134-50
Vanderklish P W, Edelman G M. 2002. Dendritic spines elongate after stimulation of group 1 metabotropic glutamate receptors in cultured hippocampal neurons. Proc Natl Acad Sci USA 99: 1639-44
Weiler I J, Greenough W T. 1993. Metabotropic glutamate receptors trigger postsynaptic protein synthesis. Proc Natl Acad Sci USA 90: 7168-71
Weiler I J, Spangler C C, Klintsova A Y, Grossman A W, Kim S H, et al. 2004. From the Cover: Fragile X mental retardation protein is necessary for neurotransmitter-activated protein translation at synapses. Proc Natl Acad Sci USA 101: 17504-9
Wong R K, Chuang S C, Bianchi R. 2002. Metabotropic Glutamate Receptors and Epileptogenesis. Epilepsy Curr 2: 81-5
Zhao W, Bianchi R, Wang M, Wong R K. 2004. Extracellular signal-regulated kinase ½ is required for the induction of group I metabotropic glutamate receptor-mediated epileptiform discharges. J Neurosci 24: 76-84
Zho W M, You J L, Huang C C, Hsu K S. 2002. The group I metabotropic glutamate receptor agonist (S)-3,5-dihydroxyphenylglycine induces a novel form of depotentiation in the CA1 region of the hippocampus. J Neurosci 22: 8838-49
Zifkin B G, Cracco R Q. 1990. An orderly approach to the abnormal EEG. In Current Practices in Clinical Electroencephalography, ed. D D Daly, T A Pedley, pp. 253-67. New York: Raven

The teachings of all of the references cited herein are hereby incorporated by reference in their entirety.

Equivalents

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating a subject, comprising the step of administering to a subject having an obsessive compulsive disorder at least one compound that down regulates Group I mGluR signaling.

2. The method of claim 1, wherein the subject is a human.

3. The method of claim 1, wherein the compound is a Group I mGluR antagonist.

4. The method of claim 1, wherein the compound down regulates mGluR1.

5. The method of claim 4, wherein the compound is an mGluR1 antagonist.

6. The method of claim 1, wherein the compound down regulates mGluR5.

7. The method of claim 6, wherein the compound is an mGluR5 antagonist.

8. The method of claim 1, wherein the subject has fragile X syndrome.

9. The method of claim 1, wherein the compound interferes with binding of a ligand to a Group I mGluR.

10. The method of claim 1, wherein the compound interferes with G-protein activation.

11. The method of claim 1, wherein the compound alters cAMP-dependent protein kinase.

12. The method of claim 1, wherein the compound alters a protein kinase.

13. The method of claim 1, wherein the compound decreases glutamate release.

14. The method of claim 1, wherein the compound is an agonist of at least one member selected from the group consisting of a Group II mGluR agonist and a Group III mGluR agonist.

15. A method of treating a subject, comprising the step of administering to a subject having an obsessive compulsive disorder at least one compound that down regulates endocannabinoid signaling.

16. The method of claim 15, wherein the compound inhibits presynaptic CB1 receptor signaling.
